(19) United States
(12) Patent Application Publication
(10) Pub. No.: US 2022/0054304 A1
(43) Pub. Date: Feb. 24, 2022

(54) SYSTEMS AND METHODS FOR PROVIDING A CARDIAC ASSISTANCE ECOSYSTEM

(71) Applicant: Cube Recovery Company, Stamford, CT (US)

(72) Inventor: Aaron Young, Stamford, CT (US)

(21) Appl. No.: 17/520,479

(22) Filed: Nov. 5, 2021

Related U.S. Application Data
(63) Continuation of application No. 16/104,674, filed on Aug. 17, 2018.
(60) Provisional application No. 62/546,696, filed on Aug. 17, 2017.

(51) Int. Cl. A61F 7/00

(52) U.S. Cl. CPC A61F 7/00; A61F 2007/002; A61F 2007/0023; A61F 2007/0002

(57) ABSTRACT

Disclosed is a cooling device, including a housing having a wall; a reservoir within the housing; a cooling material such as a liquid or gel in the reservoir; a pump within the housing; and a conduit passing adjacent the wall and through which the material is circulated by the pump from the reservoir back to the reservoir.

### RELATED PATENT APPLICATION

The application claims priority to U.S. Provisional Patent Application No. 62/546,696 filed Aug. 17, 2017, the content of which is incorporated herein by reference in its entirety.

### BACKGROUND OF THE INVENTION

Known technology is lacking in providing a soothing environment for the human heart. The systems and methods of the invention address various shortcomings of known systems.

### SUMMARY OF THE INVENTION

The invention includes devices and corresponding methods for assisting in the health of a user's heart and other body systems and components. In particular, the systems and methods of the invention provide a cardiac assistance ecosystem. Further, the systems and methods of the invention provide a cooling system which cools one or more portions of a user's body, based on a variety of input.

### BRIEF DESCRIPTION OF THE DRAWINGS

The present invention can be more fully understood by reading the following detailed description together with the accompanying drawings, in which like reference indicators are used to designate like or similar elements, and in which:

FIG. 1 is a diagram showing a central signal system 1000, in accordance with one embodiment of the invention.

FIG. 2 is a diagram showing further features of the central signal system 1000, in accordance with one embodiment of the invention.

FIG. 3 is a diagram that, in particular, shows further features of the “firmware” 110 housed within the control device 100, in accordance with one embodiment of the invention.

FIG. 4 is a further diagram showing additional particulars of the arrangement of FIG. 3, in accordance with one embodiment of the invention.

FIG. 5 is a diagram showing further details of a switch gadget, in accordance with one embodiment of the invention.

FIG. 6 is a diagram showing a cooling device controller 540 disposed upon the firmware 110, in accordance with one embodiment of the invention.

FIG. 7 is a diagram showing an example of a cooling device 510, in accordance with one embodiment of the invention.

FIG. 8 is a high-level flowchart showing aspects of processing that is performed to control the transducers (210, 220, 230), in accordance with one embodiment of the invention.

FIG. 9 is a flowchart showing further details the “perform processing to control NE transducer 210” step 810 of FIG. 8, in accordance with one embodiment of the invention.

FIG. 10 is a flowchart showing in further detail “determine if signal output (to navel entrainer (NE) transducer) is dictated by processing of firmware” step of FIG. 9, in accordance with one embodiment of the invention.

FIG. 11 is a flowchart showing in further detail “determine if signal output (to navel entrainer (NE) transducer) is dictated by processing of NE signal generator (NESCr) 330” step of FIG. 9, in accordance with one embodiment of the invention.

### DETAILED DESCRIPTION OF THE INVENTION

Hereinafter, aspects of the invention in accordance with various embodiments will be described. As used herein, any term in the singular may be interpreted to be in the plural, and alternatively, any term in the plural may be interpreted to be in the singular.

The invention includes devices and corresponding methods for assisting in the health of a user's heart and other body systems and components.

The human heart functions as a self protecting mechanism. However, the heart's ability to protect itself, on its own, is limited. The systems and methods of the invention ease strain on the heart, in accordance with embodiments of the invention. The invention protects the heart using novel environmental controls. In particular, the invention provides devices to improve the lives of people with heart related conditions, such as stress. Further, in accordance with one embodiment of the invention, the system of the invention provides a non-invasive approach to providing regulatory assistance to the cardiovascular health of a person.

The Central Signal System (CSS) of the Invention

The systems and methods of the invention provide what is herein characterized as a personalized circadian harmonic entrainment. In particular, a system of the invention uses heart “beat” based acoustic vibration to assist in the non-invasive regulation of the heart. Illustratively, the acoustic vibration may be generated using electromagnetic coils or transducers, such as at a frequency of 1 to 200 Hz, for example. The acoustic vibration may be generated using devices of the invention as is described further below. The acoustic vibration may be performed with or without time automation, sensor automation, and/or biofeedback automation, for example.

Hereinafter, further aspects of the method of the invention will be described. Centered on the biological axis between the eyes and ears, where the mouth breathes, the spine coils and reproductive organs unite, the CSS of the invention generates precision vibrations. These precision vibrations harmonize a centering rhythm to regulate and/or calibrate what is understood to be a total human compass of energy. This compass of energy may be characterized as “flux.” It is appreciated that this “flux” is not limited to the very center, front position, or back position of a human user. Rather, such compass of energy may extend to various other portions of the human body.

Illustratively, the invention may use one or more of three embodiments for the environmental regulation or entrainment of signals provided to the human body, in accordance with embodiments of the invention. These three embodiments may include a precision set biomarker from sensors associated with the user; a transducer utilized by the system of the invention; and/or direct regulation of the signals. The system of the invention may include independent or single transducer embodiments for noninvasive healing of a specific bodily region. Also, the invention may include interdependent multi-transduction regulation of one central system of the invention. Such one central system may be provided with or without time or control sensor control and/or biofeedback automation. In a manner as described further below, the signals generated by the device of the invention may be focused along the center of the human body, above and below the heart at equidistance.

The systems of the invention may include various components. One component is the “optic path entrainer” signal generator. The optic path entrainer signal generator generates signals that are directed at the optic nerves, hypothalamus pineal glands, and/or the cerebellum, for example. The invention may also include a heart harness. The heart harness may generate signals directed at the front and back of a user's heart. Such heart harness may be utilized with or without acoustic reflector material. Lastly, the invention may include a “navel entrainer signal generator.” The navel entrainer signal generator may be directed at a user's bladder or a user's spine, for example. The generated signal or transduction may be provided equidistant as the “optic entrainer” from the user's heart. The navel entrainer signal generator of the invention may (or may not) be provided with an auto-direction transducer for the programmable aim of a single transducer, adjusting automatically dependent on bodily position with or without sensors.

Hereinafter, further features of the optic path entrainer signal generator, the heart harness, and the navel entrainer signal generator will be described, in accordance with embodiments of the invention.

The navel entrainer signal generator may be provided with a “transducer C”. Stretchable material straps may be utilized to position the transducer C upon the lumbar area of the user, for example. The transducer C may be provided with input sensors. Illustratively, such input sensors may include a temperature sensor and a pressure sensor. Further, the transducer C may operate utilizing a timer, so as to regulate generated signals in a timed manner. Additionally, the transducer C may operate utilizing a photocell sensor. Such photocell sensor may sense the amount of ambient light in which the user is disposed. Based on the sensing of the ambient light, the signals may be generated in some appropriate manner. For example, if the ambient light is very low, such as would be observed at nighttime, the generation of the signals from the transducer C may be toned down, as compared to the situation in which normal day time ambient light is sensed. The navel entrainer signal generator may input data from a router, or other network system, so as to control the transducer C. The signal firewall in firmware is to process and send signal based on user's sensors alone, a buffer unable to be hacked and to protect the user from potential smart phone error, also to potentially gather aggregate data.

On the other hand, the heart harness of the invention may be provided with a “transducer B”. More specifically, the “heart harness” may include a suspender, harnessed top that straps over the heart and that is centered upon the chest. Transducer B may be disposed on the back of the heart (of the user) so as to be flanked by the back muscles of the user. Illustratively, transducer B may be controlled utilizing a timer mechanism, a Bluetooth communication protocol, photocell sensor, HRV (heart rate variability) sensor, as well as a microphone disposed on the chest of the user. Such microphone may be utilized for data input and/or medication based on such data input. It is appreciated that such transducer B could indeed be two transducers. That is, the suspender, harness top may support such two transducers such that one transducer is disposed in front of the heart of the user, and one transducer is disposed in back of the heart of the user.

Thirdly, the optic path entrainer signal generator may be in the form of a headband or hood to support a transducer A. Such headband or hood may be provided with a shiftable top so as to accommodate different hair situations and/or audio headphones, for example. Accordingly, the transducer A may be disposed on the forehead of the user. The transducer A may be controlled utilizing a photocell sensor, for example. Additionally, the transducer A may be provided and used in conjunction with a microphone utilized for data input and/or communication. Additionally, the optic path entrainer signal generator and/or transducer A may utilize Bluetooth or other suitable communication protocols. Further, each of the transducers may be detachable so as to facilitate battery replacement. In accordance with one embodiment of the invention, the operation of transducer A may utilize a timer, photocell, and/or Heart Rate Variability or “HRV” monitor, that is a Heart Rate monitor for analysis of user's heart with signal applied based on Heart Rate Variability. For example, user's “HRV” data could quantify the ideal time of day that is most beneficial to the user. Additionally, the transducer A and optic path entrainer signal generator may utilize what is herein characterized as “sleep autopilot”. Sleep autopilot may be activated manually, by the user, while utilizing other input observed by the optic path entrainer signal generator. Such other input observed by the optic path entrainer signal generator may include “yawn” detection (for example), that is detected with various assistance software that is synced to set points or biomarkers such as user's monthly HRV or sleep data for recorded signal system of firmware.

Hereinafter, further aspects of the invention will be described relating to peripheral components of the CSS. Such peripheral components of the CSS may include, but are not limited to, a responsive cooling system (RCS).

A sensor-based programmable body cooling system may be worn by a user to circulate a gentle rush of increasing coldness to the body. This soothes the body and assists the heart's relationship to heat or inflammation. The mechanism may also be utilized to “ice” an injured or over-exercised knee, ankle, shoulder, hip, wrist, elbow and/or face, for example. The mechanism may be turned on and off manually, for programmable periods of time, automatically with sensors mentioned above or pressure sensors that measure expansion/inflammation, for example.

The RCS of the invention may be powered by a rechargeable battery housed on the side of the rib cage, lumbar, or belt area, for example. The battery may be stored adjacent to, and work directly in support of, a cold source. Such cold source might be liquid or gel frozen in the form of a cube or cylindrical pack. Various further components may be utilized to incite an increasingly colder rush to areas of the body. These further components may include but are not limited to a breathable metallic layer, utilization of a sweat trap or fat deposit layer, thermometer reflector vents and/or battery cell gel lining. The RCS of the invention may circulate cooling fluid utilizing pumps. Also, the RCS of the invention may utilize electrochemical mechanisms so as to support the cooling of various fat deposits, cooling under the arms, cooling in the lumbar region, cooling in the carotid artery path, as well as cooling in the area of the heart, chest, back, neck, brainstem, and/or other portions of the cranium. The cooling supplied by the RCS may be applied in various manners including utilizing observed temperatures and applying one or more continuous flows of material so as to control temperature. The RCS of the invention may control temperature in a manual or programmable manner so as to monitor and precisely control temperature is desired.

An embodiment of the RCS may utilize sensors associated with the CSS. The RCS and/or the CSS may be powered by AC, battery or dual batteries charged in a refrigerated charger that function as AC power, sending alternating currents of coldness, decreasing potential hindrance of battery liability and/or warmth. The CSS may utilize a battery cell gel lining that serves as a reagent to release a charge that cools in an increasing manner under programmable control or manual control. The coolant material that is utilized may be controlled so as to not release charge (i.e. not cool) or be rendered active so as to release charge (i.e. to cool) Such control may be applied utilizing sensor systems, such as the sensor systems associated with the CSS. Accordingly, the systems and methods of the invention may send subtle pumps of coolant charge to fat deposits, carotid arteries, the heart and/or the brain, for example.

The systems and methods of the invention may also include “orthopedic cell regenerators.” Such orthopedic cell regenerators may utilize a set of biomarkers that control the application of acoustic signals to the human body upon the observation of predetermined bio conditions. In addition, or alternatively, such orthopedic cell regenerators may utilize a preprogrammed acoustic signal delivery system. The signals may be strategically delivered to inflamed orthopedic cells or attendant baroreceptors for injury relief with and/or without the above-described CSS, RCS, and/or orthopedic RCS components. Portions of the body, that the signals are applied to, may include the ankle, hand, some, finger, toe, knee, elbow and/or shoulder, for example, up to a complete exoskeleton i.e. one signal to each baroreceptor on body.

Hereinafter, the various further embodiments and features of the invention will be described with reference to the drawings.

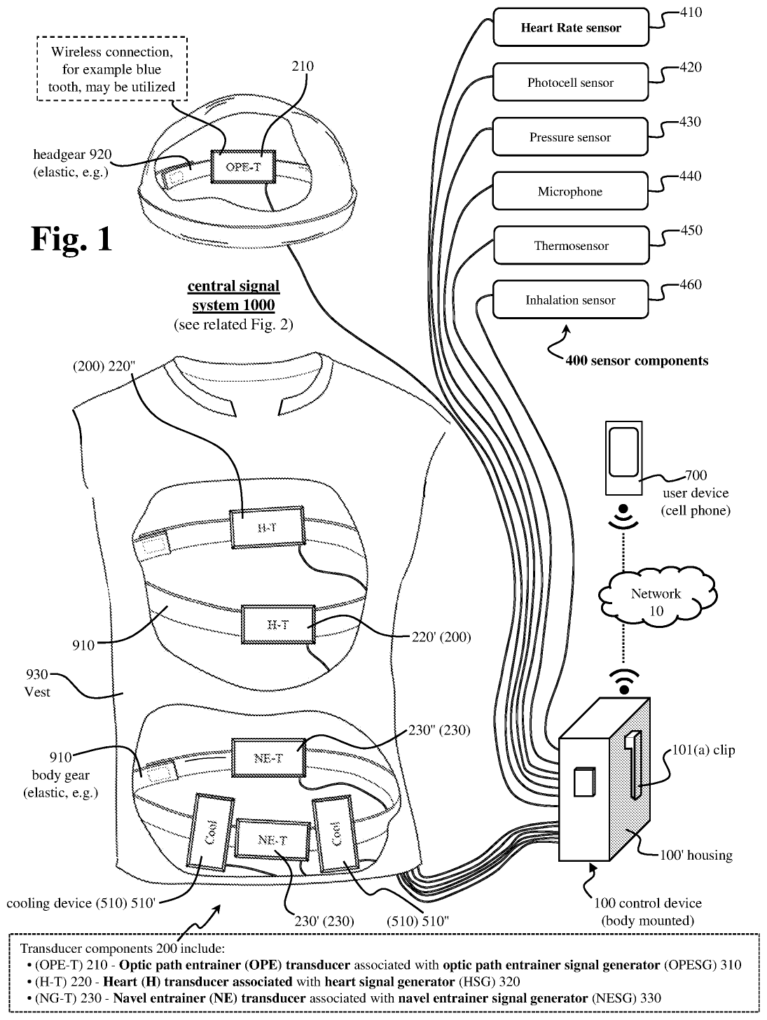

FIG. 1 is a diagram showing a central signal system 1000, in accordance with one embodiment of the invention. As shown, the central signal system 100 includes, in particular, various transducers 200, sensor components 400, and a control device 100. The central signal system 100 also includes a plurality of cooling devices (510′, 510″), collectively referred to by reference 510. As is shown, the central signal system 100 is connected to a network 10. Further, the control device 100 may be in communication (via the network 10) with a user device 700, such as a cellular phone, for example.

As described below, the control device 100 controls the transducer components 200 and/or the cooling devices 510 based on various input including data input from the sensor components 400 and/or from the user device 700.

As shown, the transducer components 200 include:

an (OPE-T) 210, i.e. an optic path entrainer (OPE) transducer, associated with optic path entrainer signal generator (OPESG) 310;

a first (H-T) 220′ and a second (H-T) 220″, i.e. a heart (H) transducers, (collectively referred to by reference 220) associated with heart signal generator (HSG) 320; as well as

a first (NG-T) 230′ and a second (NG-T) 230″, i.e. navel entrainer (NE) transducers, (collectively referred to by reference 230) associated with navel entrainer signal generator (NESG) 330.

Each of the transducers 200 may be held in place adjacent to user's body in a suitable manner. Illustratively, as shown in FIG. 1, the transducer 210 is supported by headgear 920. The headgear 920 might be in the form of an elastic band disposed within the structure of a cap or hat, for example. The ends of such an elastic band may be provided with hook and loop fasteners, such as Velcro, so as to provide adjustment.

Also, the transducers 220 and the transducers 230, as well as the cooling devices 510, may be supported by suitable body gear, such as body gear 910 shown in FIG. 1. The body gear may be in the form of elastic bands that are supported by and/or held in position by a supporting structure such as a vest 930, shirt, or some other support structure.

The various transducer components 200, as shown in FIG. 1, as well as the cooling devices 510 (including cooling device 510′ and cooling device 510″), are shown as connected to the control device 100 via physical wires. However, it is appreciated that the invention is of course not limited to a physical connection utilizing wires. Other communication channels may well be utilized, such as Bluetooth or radio communication, for example.

The central signal system 1000 includes sensor components 400, as described above. More specifically, as illustrated in FIG. 1, the sensor components may include a heartrate sensor 410, a photocell sensor 420, a pressure sensor 430, a microphone 440, a thermal sensor 450, and/or an inhalation sensor 460.

It is appreciated that not all of the sensor components 400 may be compatible with and/or utilized with each of the transducer components 200 and/or the cooling devices 510. Further details of the particular processing of the sensor components 400 in conjunction with the transducer components 200 and cooling devices 510 are described below.

As shown in FIG. 1, the control device 100 is physically manifested in a housing 100′. The control device 100 may be provided with a mechanical arrangement to attach the control device 100 upon the body of a user, such as the clip 101(a) as shown. However, it is appreciated that the systems and methods of the invention are not limited to the particular arrangement shown in FIG. 1. Indeed, the housing 100′ may be integrated into one and the same housing as one of the transducers shown, for example. Also, the housing 100′ may be integrated into one and the same housing as one of the cooling devices 510 shown. In such a situation, where the control device 100 is integrated into a cooling device and/or a transducer, the controlling device 100 may then communicate with other transducers and/or cooling devices—so as to control such other transducers and/or cooling devices. This communication might be through a wire communication, a wireless communication such as Bluetooth, and/or some other communication channel, as desired.

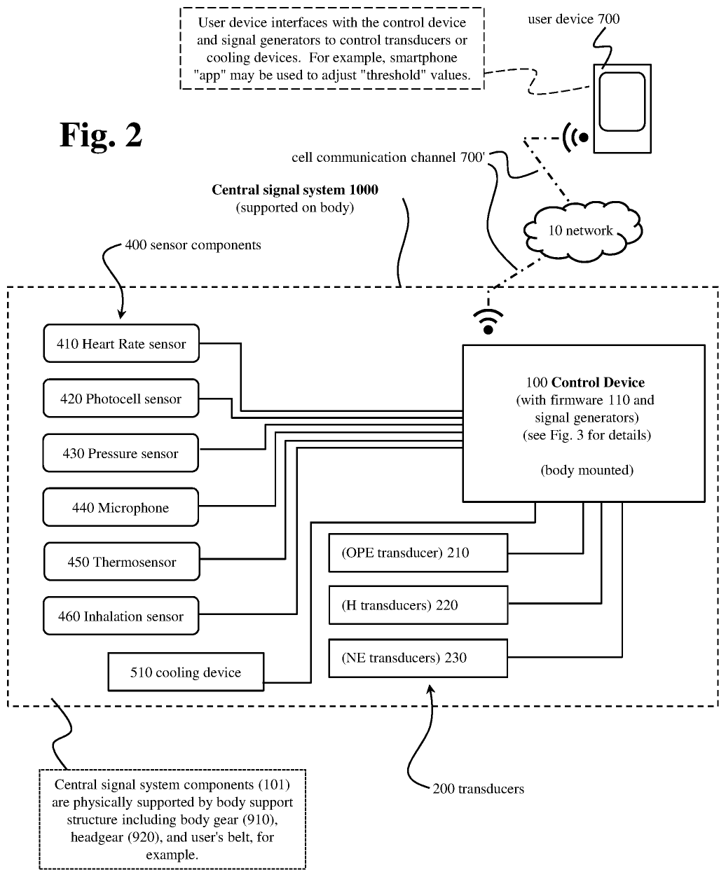

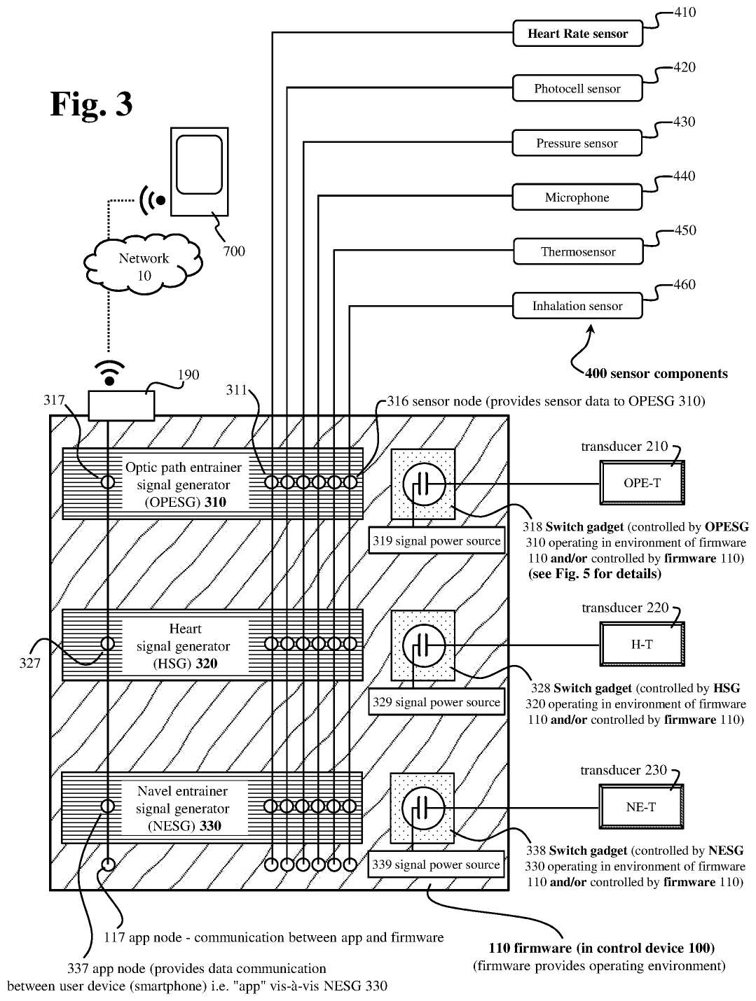

FIG. 2 is a diagram showing further features of the central signal system 1000, in accordance with one embodiment of the invention. As shown, the central signal system 1000 includes a plurality of sensors, a plurality of transducers, and a cooling device. Further, the central signal system 1000 includes the control device 100. Further details of the control device 100 are described below with reference to FIG. 3. In particular, the control device 100 may include firmware 110 as well as a plurality of signal generators. As noted above, the control device 100, as well as the transducers and/or cooling devices, may be mounted on the body of the user.

In similar manner as is represented in FIG. 1, FIG. 2 reflects that the control device 100 may communicate with a user device 700 via a suitable network 10 For example, the network 10 might be a cell communication channel 700′. FIG. 2 also reflects that the user device may interface with the control device, as well as signal generators (described below), to control the transducers and/or cooling devices. For example, a smart phone “app” may be used to adjust “threshold” values. It is appreciated that such feature is merely an example, and a smart phone may be utilized to control the transducers and/or cooling devices in various other ways and using various methodologies.

FIG. 3 is a diagram that, in particular, shows further features of the “firmware” 110 housed within the control device 100, in accordance with one embodiment of the invention. The firmware 110 may be in the form of a set of instructions (disposed on a computer readable medium) that provides an operating system, i.e. an operating environment. As shown in FIG. 3, disposed upon (and/or in the environment) of the firmware 110) is an optic path entrainer signal generator 310, a heart signal generator 320, and a navel entrainer signal generator 330. In accordance with one embodiment of the invention, each of the signal generators operate in the operating environment provided by the firmware 110. In such environment, the signal generators may be separate processing components and/or the signal generators may be constituted by separate sets of computer instructions disposed on the same computer readable medium as the firmware, for example, in accordance with embodiments of the invention. It is appreciated that various variations of such arrangement are within the scope of the invention, as described further below.

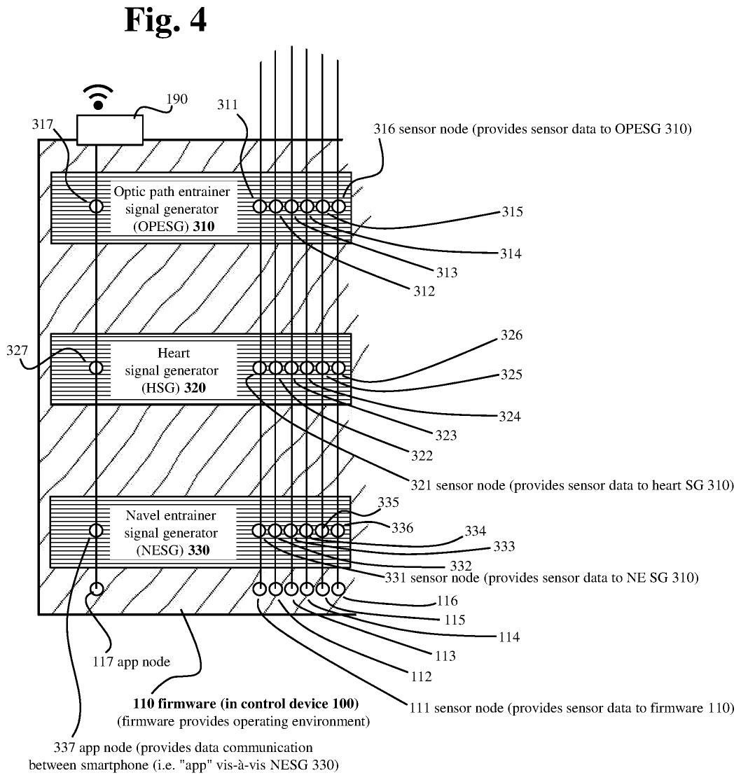

FIG. 4 is a further diagram showing additional particulars of the arrangement of FIG. 3, in accordance with one embodiment of the invention.

As shown in FIG. 3, each of the signal generators may utilize similar processing architecture. Accordingly, the signal generator 310 will hereinafter be described—with the understanding that the signal generator 320 and the signal generator 330 may utilize similar arrangements.

Various aspects of the processing performed by the signal generator 310 are described below. As shown in FIG. 3 (and shown more specifically in FIG. 4) the signal generator 310 includes a plurality of sensor nodes (311, 312, 313, 314, 315, 316). Each of such sensor nodes provides a communication channel by which the signal generator 310 may input data, respectively, from the sensor components 400. Accordingly, the sensor node 311 provides a communication channel by which the signal generator 310 may communicate with the heart rate sensor 410. Further, the sensor node 312 provides a communication channel by which the signal generator 310 may communicate with the photocell sensor 420. The sensor node 313 provides a communication channel by which the signal generator 310 may communicate with the pressure sensor 430. The sensor node 314 provides a communication channel by which the signal generator 310 may communicate with the microphone 440. The sensor node 315 provides a communication channel by which the signal generator 310 may communicate with the thermal sensor 450. Lastly, the sensor node 316 provides a communication channel by which the signal generator 310 may communicate with the inhalation sensor 460. Accordingly, the plurality of sensor nodes (311, 312, 313, 314, 315, 316) respectively provide data to the signal generator 310. Based on this input data (as well as other communicated data), signal generator may perform a variety of processing as is otherwise described herein.

As shown in FIG. 3, the signal generator 310 also includes what is herein characterized as an “app” node 317. The app node 317 provides data communication between the signal generator 310 and the user device 700, such as a smart phone 700. Accordingly, the signal generator 310 may output data to the user device 700 via the app node 317. Also, the signal generator 310 may input data from the user device 700 via the app node 317. In the embodiment shown in FIG. 3, this communication may be performed through a communication portion 190 which interfaces with network 10, and in turn the user device 700. The signal generator 320 may similarly be provided with an app node 327 The signal generator 330 may also similarly be provided with an app node 337.

Additionally, the architecture may include an app node 117. The app node 117 provides communication between the firmware, itself, and the user device 700. Further, as shown in FIG. 3 and in FIG. 4, the architecture may include a plurality of sensor nodes (111, 112, 113, 114, 115, 116) that provides sensor data to the firmware 110. In other words, such sensor nodes (111, 112, 113, 114, 115, 116) provide a communication channel by which the firmware 110 may, respectively, communicate with each of the sensor components. Accordingly, various input data from the various sensor components may be utilized by the firmware 110 acting independently, the signal generators acting independently (within the operating environment of the firmware 110) and/or the firmware 110 acting in conjunction with one or more of the signal generators.

As shown in FIG. 3, the architecture of FIG. 3 further includes what is herein characterized as a switch gadget 318. The switch gadget 318 is the mechanism by which the signal generator 310 and/or the firmware 110 controls the transducer 210. More specifically, in accordance with one embodiment of the invention, the switch gadget 318 is a mechanism by which a signal power source 319 may be connected or disconnected with the transducer 210. In one embodiment, the switch gadget 318 may simply be in the form of a mechanical switch that is opened or closed (by the switch gadget 318) to either connect the signal power source 319 to the transducer 210, or alternatively, disconnect the signal power source 319 to the transducer 210. However, the invention is not limited to such basic switch. Rather, it is appreciated that the switch gadget 318 may include any of the variety of arrangements so as to control power flowing from a signal power source 319 to the transducer 210. Accordingly, the switch gadget 318 may control various aspects of the power running between the signal power source 319 to the transducer 210. Such aspects may include the level of power, the periodicity of power pulses, the duration of power pulses, frequency attributes of the pulses, and/or other attributes of power running from the signal power source 319 to the transducer 210.

As shown in FIG. 3, in similar manner, the arrangement includes a switch gadget 328. The switch gadget 328 is controlled (by the signal generator 320, by the firmware 110, or by the signal generator 320 working in conjunction with the firmware 110) to control the passage of power from a signal power source 329 to the transducer 220.

As further shown in FIG. 3, in similar manner, the arrangement includes a switch gadget 338. The switch gadget 338 is controlled (by the signal generator 330, by the firmware 110, or by the signal generator 330 working in conjunction with the firmware 110) to control the passage of power from a signal power source 339 to the transducer 230.

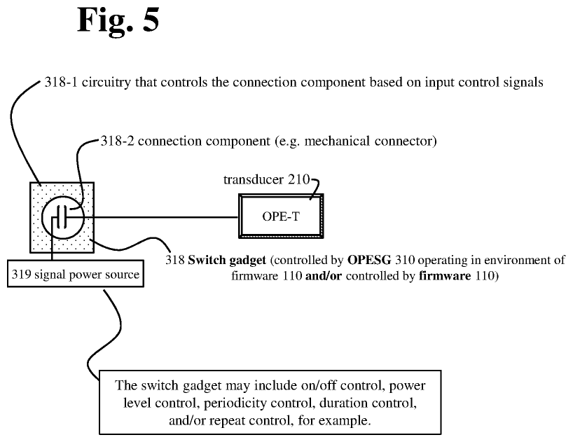

FIG. 5 is a diagram showing further details of a switch gadget, in accordance with one embodiment of the invention. In particular. FIG. 5, as an illustrative example, shows further details of the switch gadget 318. As described above, the switch gadget 318 controls the transfer of power between the signal power source 319 and the transducer 210 The switch gadget 318 is controlled by the signal generator 310 operating in the environment of the firmware 110 and/or controlled by the firmware 110, or even alternatively, controlled by both the signal generator 310 and the firmware 110, working in conjunction with each other. The switch gadget 310 includes circuitry 318-1 that controls a suitable connection component, i.e., so as to connect or disconnect the signal power source 319 to the transducer 210. This connection is performed based on input control signals from the signal generator 310 and/or firmware 110. Relatedly, the switch gadget 318 includes the connection component 318-2. In its simplest form, the connection component 318-2 might simply be in the form of a mechanical switch that opens/closes utilizing a suitable actuator, for example. As reflected in FIG. 5, the switch gadget may include on/off control, power level control, periodicity control, duration control, and/or repeat control, for example.

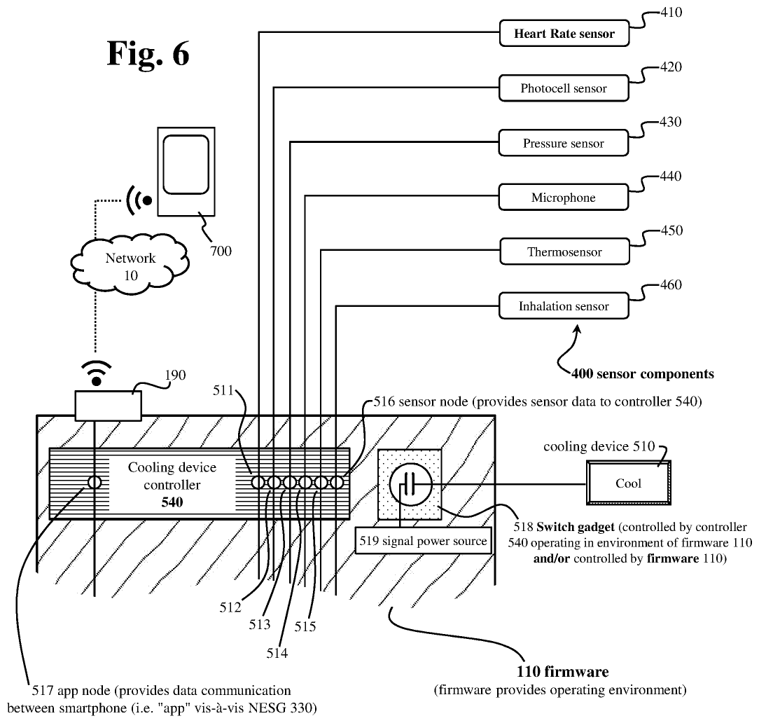

In addition to the signal generators disposed upon the firmware 110, as described above, a cooling device controller 540 may also be disposed upon and/or operate in the environment provided by the firmware 110. FIG. 6 is a diagram showing such cooling device controller 540 disposed upon the firmware 110, in accordance with one embodiment of the invention.

In such environment, the cooling device controller may be a separate processing component and/or may be constituted by separate set(s) of computer instructions disposed on the same computer readable medium as the firmware, for example. It is appreciated that various variations of such arrangement are within the scope of the invention, as described further below.

As shown in FIG. 6, the cooling device controller includes a plurality of sensor nodes (511, 512, 513, 514, 515, 516). Each of such a sensor nodes provides a communication channel by which the cooling device controller may input data, respectively, from the sensor components 400. Accordingly, the sensor node 511 provides a communication channel by which the cooling device controller 540 may communicate with the heart rate sensor 410. Further, the sensor node 512 provides a communication channel by which the cooling device controller may communicate with the photocell sensor 420. The sensor node 513 provides a communication channel by which the cooling device controller may communicate with the pressure sensor 430. The sensor node 514 provides a communication channel by which the cooling device controller may communicate with the microphone 440. The sensor node 515 provides a communication channel by which the cooling device controller may communicate with the thermal sensor 450. Lastly, the sensor node 516 provides a communication channel by which the cooling device controller 540 may communicate with the inhalation sensor 460. Accordingly, the plurality of sensor nodes (511, 512, 513, 514, 515, 516) respectively provide data to the cooling device controller. Based on this data (as well as other communicated data), the cooling device controller may perform a variety of processing as is otherwise described herein.

As shown in FIG. 6, the cooling device controller 540 also includes what is herein characterized as an “app” node 517. The app node 517 provides data communication between the cooling device controller 540 and the user device 700, such as a smart phone 700. Accordingly, the cooling device controller 540 may output data to the user device 700 via the app node 517. Also, the cooling device controller 540 may input data from the user device 700 via the app node 517. In the embodiment shown in FIG. 6, this communication may be performed through the communication portion 190 which interfaces with network 10, and in turn the user device 700.

As shown in FIG. 6, the architecture of FIG. 6 further includes the switch gadget 518. The switch gadget 518 is the mechanism by which the cooling device controller 540 and/or the firmware 110 controls the cooling device 510. More specifically, in accordance with one embodiment of the invention, the switch gadget 518 is a mechanism by which a power source 519 may be connected or disconnected with the cooling device 510. In one embodiment, the switch gadget 518 may simply be in the form of a mechanical switch that is opened or closed (by the switch gadget 518) to either connect the power source 519 to the cooling device 510, or alternatively, disconnect the signal power source 3192 the transducer 210. However, the invention is not limited to such basic switch. Rather, it is appreciated that the switch gadget 518 may include any of the variety of arrangements so as to control power flowing from the power source 519 to the cooling device 510. Accordingly, the switch gadget 518 may control various aspects of the power running between the power source 519 to the cooling device 510. Such aspects may include the level of power, the periodicity of power pulses, the duration of power pulses, frequency attributes of the pulses, and/or other attributes of power running from the power source 519 to the cooling device 510.

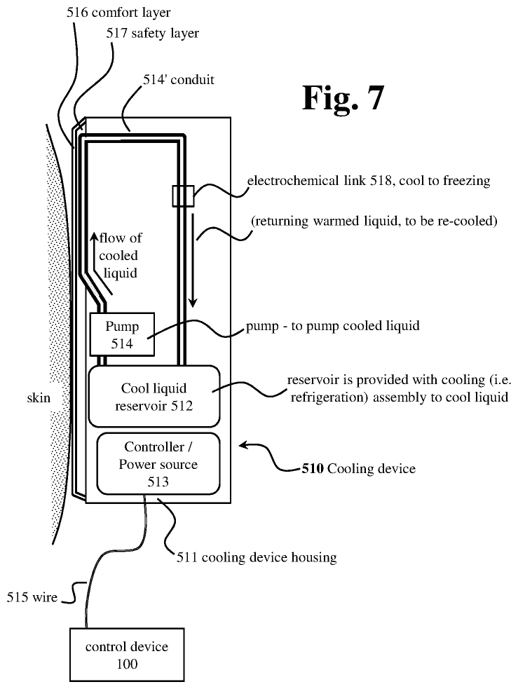

As described above, the systems and methods of the invention may include a cooling device, such as the cooling device 510 shown in FIG. 6. FIG. 7 is a diagram showing an example of a cooling device 510, in accordance with one embodiment of the invention. As shown, the cooling device 510 includes a cooling device housing 511. For example, the cooling device housing 511 might be constructed of plastic so as to enclose and support the interior components of the cooling device 510. Within the housing, is a cool liquid reservoir 512. The cool liquid reservoir 512, in accordance with one embodiment of the invention, contains a cool liquid that is circulated within the cooling device 510 through conduit 514′. The circulation of such cool liquid is provided by a pump 514. A controller/power source 513 is provided to control the pump. More specifically, the controller/power source 513 may be controlled (as described above) to apply varying amounts of coolness to the skin. In the example of FIG. 7, the particular level of coolness may be controlled by varying the circulation of the cool liquid. That is, in operation, newly cooled liquid is pumped, by pump 514, so as to be adjacent to the skin. Upon that liquid being warmed, as a result of being adjacent the skin, such warmed liquid is then replaced, via pumping, with additional newly cooled liquid. Hand-in-hand, the warmed liquid is then returned to the cool liquid reservoir—so as to be re-cooled. The cooling device 510 may include, as shown, an electrochemical link 518, or other arrangement, to control the cooling of the liquid to close to the freezing point, or other desired temperature. For example, the cool liquid reservoir 512 may be provided with a cooling assembly to cool the liquid, such as a refrigeration arrangement. The cooling device may be provided with a comfort layer 516 and a safety layer 517, such as to prevent frost-bite or other adverse condition from occurring to the user.

As shown in FIG. 7 and also described above, the cooling device 510 (and specifically the controller/power source 513 of the cooling device) may be connected to the control device 100 via a wire 515 or via some other communication channel.

As described above, the cooling material utilized in the cooling device 510 is in the form of a liquid. However, the invention is not limited to such particular. Rather, it is appreciated, that the cooling material might be in the form of a gel, for example, as otherwise described herein. As with liquid, such gel might be circulated through the cooling device 510 in varying degrees—so as to provide the desired cooling.

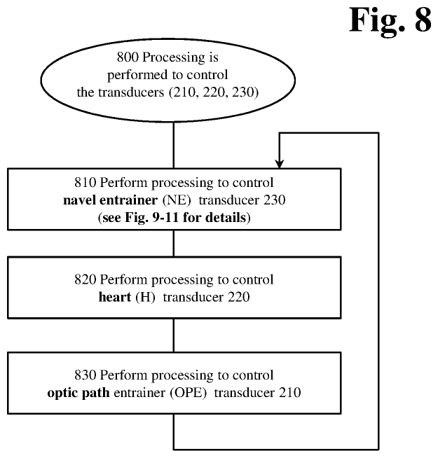

FIG. 8 is a high-level flowchart showing aspects of processing that is performed to control the transducers (210, 220, 230), in accordance with one embodiment of the invention.

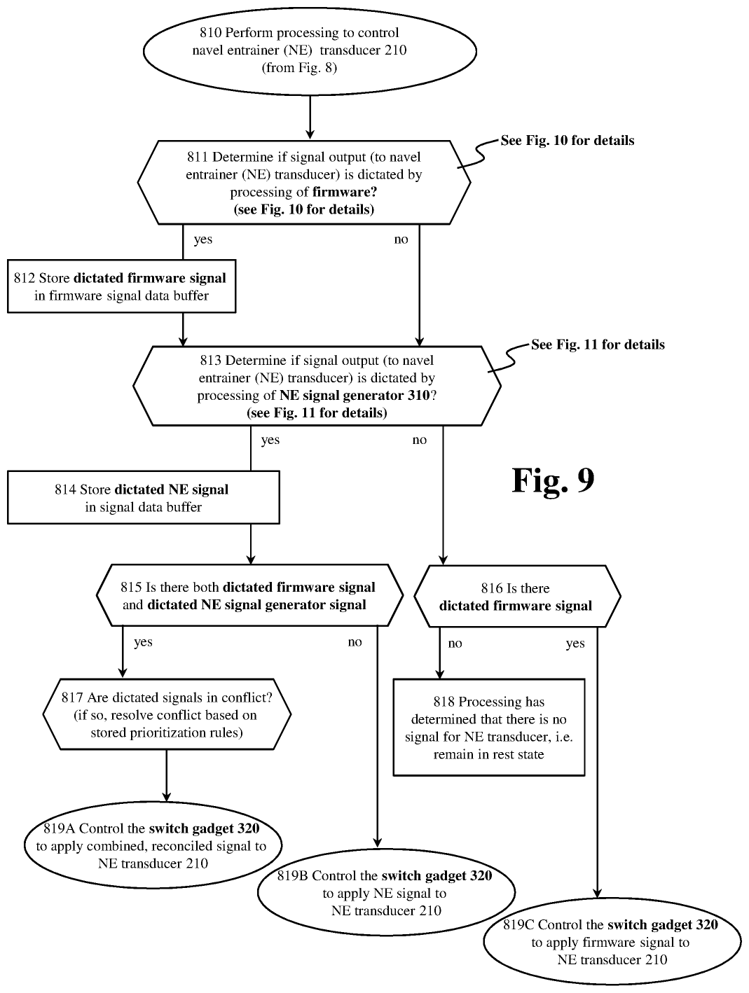

As shown, the processing starts in step 800 and passes to step 810. In step 810, the system performs processing to control the transducer 230. Further details are described below with reference to FIGS. 9-11. Then, the process passes to step 820. In step 820, processing is performed to control the heart transducer 220. Then, in step 830, processing is performed to control the transducer 210. It is appreciated that the processing of FIG. 8 shows control of the transducers being performed in a serial manner, such as may be the case with a particular processor executing computer readable instructions in a serial manner. However, it is appreciated that the processing shown in FIG. 8 may alternatively be performed using parallel processing—or performed using both serial processing and parallel processing methodologies.

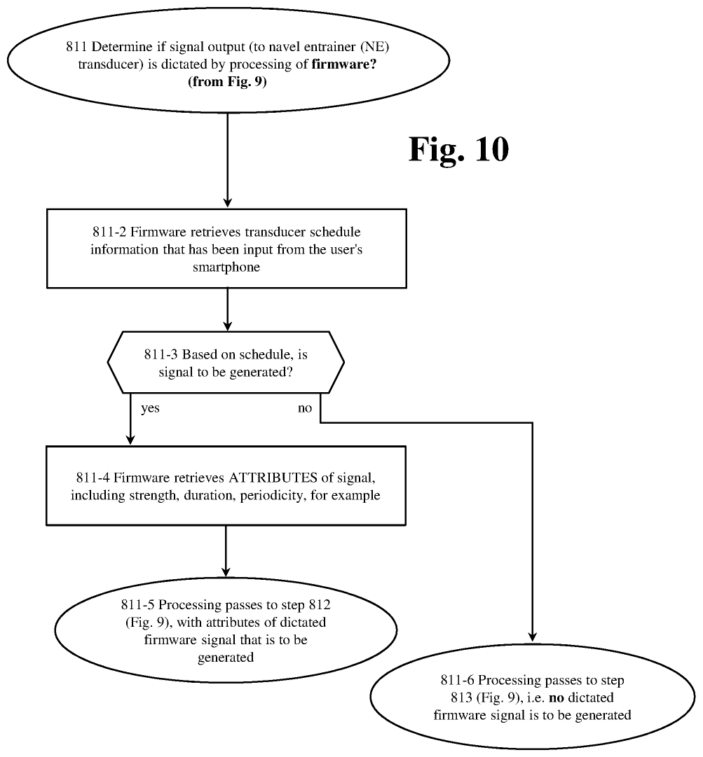

FIG. 9 is a flowchart showing further details the “perform processing to control NE transducer 210” step 810 of FIG. 8, in accordance with one embodiment of the invention. As shown, the process starts in step 810 and passes to step 811. In step 811, the process determines if a signal output, to the transducer 210, is dictated by the processing of firmware. Further details of this processing are described below with reference to FIG. 10. If yes in step 811, then the processing passes to step 812. In step 812, the processing stores the dictated firmware signal into what is herein characterized as a firmware signal data buffer. In other words, for example, the dictated firmware signal may be put into short-term memory. After the processing of step 812, the process passes to step 813. On the other hand, if no in step 811, then the processing passes directly to step 813.

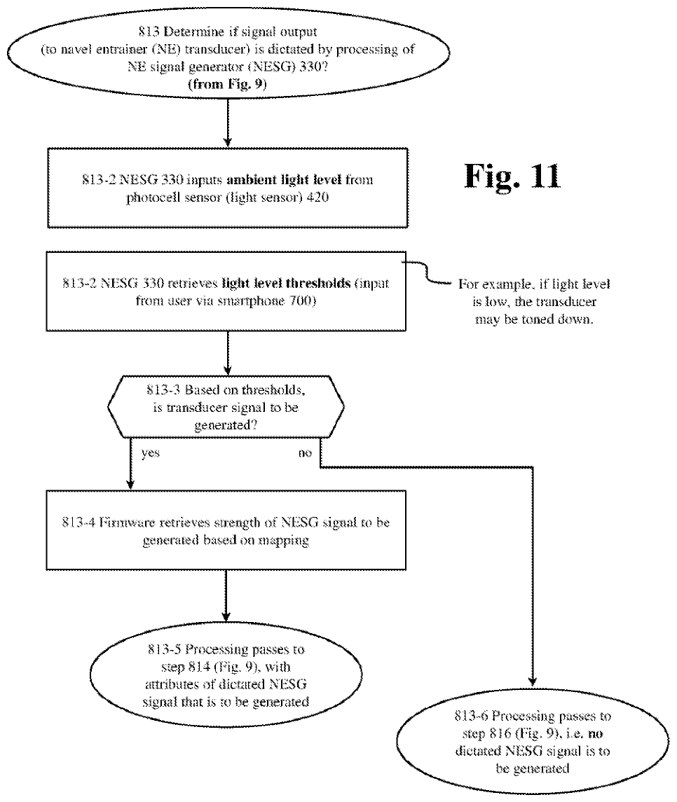

In step 813, the processing determines if the signal output to the transducer 210 is dictated by the processing of the signal generator 310. Further details of this processing are described below with reference to FIG. 11. If yes in step 813, processing passes to step 814. In step 814, the process stores the dictated NE signal in a suitable signal data buffer. In other words, the dictated NE signal is stored in a short term memory, for example. After step 814, the process passes to step 815. In step 815, the processing determines whether there is both a dictated firmware signal AND a dictated NE signal generator signal.

If yes in step 815, this reflects that both the signal generator 310 and the firmware are generating commands to control the transducer 210. Illustratively, the signal generator 310 may be controlling the strength of the signal, whereas the firmware is controlling the duration of a pulse of the signal. In this situation, there would be no conflict. However, to address the situation of the conflict, the process passes to step 817. In step 817, the processing determines whether the dictated signals are indeed in conflict. Further, step 817 (of FIG. 9) reflects that if the dictated signals are in conflict, then the processing should resolve such conflict. For example, the resolution of such conflict might be based on stored prioritization roles. For example, it might be the case that a prioritization rule dictates that, given a conflict, firmware should always take precedence.

After step 817 of FIG. 9, the process passes to step 819A. In step 819A, the processing controls the switch gadget 320 to apply the combined, reconciled signal to the NE transducer 210.

With further reference to the processing of step 815 (of FIG. 9), if no in step 815, then the processing passes to step 819B. In step 819B, the processing controls the switch gadget 320 so as to apply the NE signal to the NE transducer 210.

With further reference to step 813, it may be that the signal generator 310 has not generated any signal to control the transducer 210. In such a situation, the processing passes from step 813 to step 816. In step 816, the process determines whether there is a dictated firmware signal. This processing utilizes the stored dictated firmware signal, which was stored in step 812 above.

If it is determined that there is not a dictated firmware signal, then the processing passes to step 818. In step 818, the processing has indeed determined that there is NO signal for the NE transducer. Accordingly, the NE transducer will remain in a rest state, or be put into a rest state.

On the other hand, if there is indeed a dictated firmware signal, then the process passes from step 816 to step 819C. The processing then controls the switch gadget 320 to apply the sole signal, i.e. the firmware signal, to the NE transducer 210.

FIG. 10 is a flowchart showing in further detail the “determine if signal output (to the NE transducer) is dictated by processing of firmware” step 811 of FIG. 9, in accordance with one embodiment of the invention. The processing of FIG. 10 is provided to show an example of the manner in which the firmware might control the NE transducer.

As shown in FIG. 10, the process starts in step 811 and passes to step 811-2. In step 811-2, the firmware retrieves transducer schedule information that has been input from a user's smartphone. Then, in step 811-3, the system determines, based on a schedule, whether a signal is to be generated. For example, this might include comparing data in the schedule vis-à-vis actual observed time. If a signal is to be generated, the process passes from step 811-3 to step 811-4. In such step, the firmware retrieves attributes of the dictated signal including strength, duration, and periodicity, for example. Then, the processing passes to step 811-5. In step 811-5, the processing passes to step 812 (of FIG. 9) with the attributes of the particular dictated firmware signal that is to be generated.

On the other hand, if no in step 811-3, the processing passes to step 811-6. In step 811-6, the process passes to step 813 of FIG. 9. In other words, no dictated firmware signal is to be generated.

FIG. 11 is a flowchart showing in further detail the “determine if signal output (to navel entrainer (NE) transducer) is dictated by processing of NE signal generator (NESG)” step 813 of FIG. 7, in accordance with one embodiment of the invention. FIG. 11 shows an example of the manner in which the NE signal generator might control the NE transducer 230.

As shown, the process starts in step 813, and passes to step 813-2. In step 813-2, the NE signal generator 330 inputs an observed ambient light level from the photocell sensor (light sensor) 420. Then, in step 813-2, the NE signal generator 330 retrieves light level thresholds, which have been input, for example, from a user via the user smartphone 700. Then, the process passes to step 813-3 In step 813-3, the processing determines, based on threshold values, whether or not a transducer signal is to be generated.

If yes in step 813-3, the process passes to step 813-4. In step 813-4, the firmware retrieves the strength of the NESG signal to be generated. For example, this processing might be performed utilizing a mapping that associates a particular light level with a particular signal strength. Then, the process passes to step 813-5. In step 813-5, the processing passes to step 814 (of FIG. 9), with attributes of the dictated NESG signal that is to be generated.

On the other hand, if no in step 813-3, the processing passes to step 813-6. In step 813-6, the processing returns to step 816 of FIG. 9—thus reflecting that no signal is to be generated.

In accordance with embodiments of the invention, it is appreciated that processing to control the heart (H) transducer 220 (step 820 of FIG. 8) and processing to control the optic path entrainer (OPE)transducer 210 (step 830 of FIG. 8) may be performed, for example, in a manner similar to that illustrated in FIGS. 9-11.

Attached hereto, and a part of this provisional patent application filing, is “Attachment-A” (including pages 1-5) entitled “External Cardiac Assistance Ecosystem”.

Attachment-A also includes various description and further details in accordance with embodiments of the invention.

On page 5 of the Attachment-A is Diagram A-1 showing features of the invention, in accordance with at least one embodiment of the invention.

As shown, Diagram A-1 shows a central signal system and in particular, a central signal flow with sensors.

As shown, the central signal system includes a plurality of communicable elements, a plurality of entrainers, a user device, a sensor/signal routing matrix, firmware, and relatedly, software. The communicable elements (as characterized herein) include a transducer and likely more than one transducer, a heart rate sensor, a photocell sensor, a pressure sensor, a microphone, a thermosensor, an inhalation sensor, a Bluetooth component, a mini USB/lightning, and a responsive cooling system. The mini USB, lightning, are ports for wired compatibility—such as compatibility with Apple, Samsung, and Google phones for example—such being provided in addition to Bluetooth, in accordance with embodiments of the invention.

The plurality of entrainers include an optic path entrainer, a heart entrainer, and a navel entrainer.

The user device may be any of a wide variety of devices including a smart phone, a watch with an on-board computer, or a PC (personal computer) app i.e. application, for example. As shown, such a user device may be provided with any number of features, including voice assistance, telecommunication features, time jam features, ear audio features, one or more sensors to provide human symptom input, and suggestion output (such as an audio output that provides useful information to a user).

Such “time jam” feature relates to synching time between two devices in embodiments of the invention. The time jam feature addresses the problem that two devices' respective time codes, even if initially synched together, tend to drift by seconds without a synchronization device. The invention provides such a synchronization device that may be implemented in the form of an “app”, which is coded to monitor and synch together two devices—such as a smartphone and firmware of device—to maintain time exactness and prevent temporal drifts or desynchronization.

As shown, the firmware of the invention may also include a variety of features, including sensor input, a signal firewall, a signal biomarker set, signal source/output features, an aggregate blackbox (that collects and stores various information, i.e. data, regarding the central signal system), and may further be provided with the ability to be re-written via “factory updates” to the firmware.

The software may be disposed in the operating environment of the firmware as otherwise described herein.

The central signal system further includes the sensor/signal routing matrix, as noted above, and shown in Diagram A-I. The sensor/signal routing matrix may be provided to control various signals and/or other data moving to and from (i.e. in and out) of the various components of the central signal system Diagram A-1 includes indicia that shows, in accordance with one embodiment of the invention, a mapping of communication between the communicable elements and the various entrainers—in conjunction with the association of the communicable elements/various entrainers vis-à-vis the other components of the central signal system. As shown for example, the heart rate sensor is in communication with the heart entrainer. As shown, for example, the photocell sensor is in communication with both the optic path entrainer and the heart entrainer. As shown for example, the pressure sensor is in communication with the heart entrainer and the navel entrainer. As shown, for example, the microphone is in communication with the optic path entrainer and the heart entrainer. As shown, for example, the thermosensor is in communication with the heart entrainer and the navel entrainer. As shown, for example, the inhalation sensor is in communication with the heart entrainer and the navel entrainer. Further, channels of communication of the Bluetooth, mini USB/lightning and responsive cooling system, vis-à-vis the entrainers, are illustrated.

Each of the optic path entrainer, the heart entrainer, and the navel entrainer may be in communication with a respective transducer. On the other hand, each of the optic path entrainer, the heart entrainer, and the navel entrainer may be in communication with a same transducer, i.e. so as to collectively control such same transducer (in some suitable manner).

It is appreciated that features of a particular embodiment described herein may be used in conjunction with other embodiments, as may be desired.

Hereinafter, further aspects of implementation of the systems and methods of the invention will be described.

As described herein, embodiments of the system of the invention and various processes of embodiments are described. The system of the invention and/or portions of the system of the invention may be in the form of a specialized “processing machine,” i.e. a tangibly embodied specialized machine. As used herein, the term “processing machine,” is to be understood to include at least one “processor” that uses at least one memory. The various processing portions as described herein, may in the form of one or more “processors” in accordance with embodiments of the invention. The processing portions are associated with at least one memory that stores a set of instructions. The instructions may be either permanently or temporarily stored in the memory or memories of the processing machine. The processor executes the instructions that are stored in the memory or memories in order to process data. The set of instructions may include various instructions that perform a particular task or tasks, such as any of the processing as described herein. Such a set of instructions for performing a particular task may be characterized as a program, software program, code or simply software, for example.

As noted above, the processing machine, which may be constituted, for example, by the particular system and/or systems described above, executes the instructions that are stored in the memory or memories to process data. This processing of data may be in response to commands by a user or users of the processing machine, in response to previous processing, in response to a request by another processing machine and/or any other input, for example.

As noted above, the machine used to implement the invention may be in the form of a processing machine. The processing machine may also utilize (or be in the form of) any of a wide variety of other technologies including a special purpose computer, a computer system including a microcomputer, mini-computer or mainframe for example, a programmed microprocessor, a micro-controller, a peripheral integrated circuit element, a CSIC (Consumer Specific Integrated Circuit) or ASIC (Application Specific Integrated Circuit) or other integrated circuit, a logic circuit, a digital signal processor, a programmable logic device such as a FPGA, PLD, PLA or PAL, or any other device or arrangement of devices that is capable of implementing the steps of the processes of the invention.

The processing machine used to implement the invention may utilize a suitable operating system. Thus, embodiments of the invention may include a processing machine running the Windows 10 operating system, the Windows 8 operating system, Microsoft Windows™ Vista™ operating system, the Microsoft Windows™ XP™ operating system, the Microsoft Windows™ NT™ operating system, the Windows™ 2000 operating system, the Unix operating system, the Linux operating system, the Xenix operating system, the IBM AIX™ operating system, the Hewlett-Packard UX™ operating system, the Novell Netware™ operating system, the Sun Microsystems Solaris' operating system, the OS/2™ operating system, the BeOS™ operating system, the Macintosh operating system, the Apache operating system, an OpenStep™ operating system or another operating system or platform.

It is appreciated that in order to practice the method of the invention as described above, it is not necessary that the processors and/or the memories of the processing machine be physically located in the same geographical place. That is, each of the processors and the memories used by the processing machine may be located in geographically distinct locations and connected so as to communicate in any suitable manner. Additionally, it is appreciated that each of the processor and/or the memory may be composed of different physical pieces of equipment. Accordingly, it is not necessary that the processor be one single piece of equipment in one location and that the memory be another single piece of equipment in another location. That is, it is contemplated that the processor may be two pieces of equipment in two different physical locations. The two distinct pieces of equipment may be connected in any suitable manner, such as via a network. Additionally, the memory may include two or more portions of memory in two or more physical locations.

To explain further, processing as described above is performed by various components and various memories. However, it is appreciated that the processing performed by two distinct components as described above may, in accordance with a further embodiment of the invention, be performed by a single component. Further, the processing performed by one distinct component as described above may be performed by two distinct components. In a similar manner, the memory storage performed by two distinct memory portions as described above may, in accordance with a further embodiment of the invention, be performed by a single memory portion. Further, the memory storage performed by one distinct memory portion as described above may be performed by two memory portions.

Further, as also described above, various technologies may be used to provide communication between the various processors and/or memories, as well as to allow the processors and/or the memories of the invention to communicate with any other entity; i.e., so as to obtain further instructions or to access and use remote memory stores, for example. Such technologies used to provide such communication might include a network, the Internet, Intranet, Extranet, LAN, an Ethernet, or any client server system that provides communication, for example. Such communications technologies may use any suitable protocol such as TCP/AP, UDP, or OSI, for example.

As described above, a set of instructions is used in the processing of the invention on the processing machine, for example. The set of instructions may be in the form of a program or software. The software may be in the form of system software or application software, for example. The software might also be in the form of a collection of separate programs, a program module within a larger program, or a portion of a program module, for example. The software used might also include modular programming in the form of object oriented programming. The software tells the processing machine what to do with the data being processed.

Further, it is appreciated that the instructions or set of instructions used in the implementation and operation of the invention may be in a suitable form such that the processing machine may read the instructions. For example, the instructions that form a program may be in the form of a suitable programming language, which is converted to machine language or object code to allow the processor or processors to read the instructions That is, written lines of programming code or source code, in a particular programming language, are converted to machine language using a compiler, assembler or interpreter, for example. The machine language is binary coded machine instructions that are specific to a particular type of processing machine, i.e., to a particular type of computer, for example. The computer understands the machine language.

A suitable programming language may be used in accordance with the various embodiments of the invention. Illustratively, the programming language used may include assembly language, Ada, APL, Basic, C, C++, COBOL, dBase, Forth, Fortran, Java, Modula-2, Pascal, Prolog, REXX, Visual Basic, and/or JavaScript, for example. Further, it is not necessary that a single type of instructions or single programming language be utilized in conjunction with the operation of the system and method of the invention. Rather, any number of different programming languages may be utilized as is necessary or desirable.

Also, the instructions and/or data used in the practice of the invention may utilize any compression or encryption technique or algorithm, as may be desired. An encryption module might be used to encrypt data. Further, files or other data may be decrypted using a suitable decryption module, for example.

As described above, the invention may illustratively be embodied in the form of a processing machine, including a computer or computer system, for example, that includes at least one memory. It is to be appreciated that the set of instructions, i.e., the software for example, that enables the computer operating system to perform the operations described herein may be contained on any of a wide variety of media or medium, as desired. Further, the data that is processed by the set of instructions might also be contained on any of a wide variety of media or medium. That is, the particular medium, i.e., the memory in the processing machine, utilized to hold the set of instructions and/or the data used in the invention may take on any of a variety of physical forms, for example. Illustratively, as also described above, the medium may be in the form of paper, paper transparencies, a compact disk, a DVD, an integrated circuit, a hard disk, a floppy disk, an optical disk, a magnetic tape, a RAM, a ROM, a PROM, a EPROM, as well as any other medium or source of data that may be read by the processors of the invention.

Further, the memory or memories used in the processing machine that implements the invention may be in any of a wide variety of forms to allow the memory to hold instructions, data, or other information, as is desired. Thus, the memory might be in the form of a database to hold data. The database might use any desired arrangement of files such as a flat file arrangement or a relational database arrangement, for example.

In the system and method of the invention, a variety of “user interfaces” may be utilized to allow a user to interface with the processing machine or machines that are used to implement the invention. As used herein, a user interface includes any hardware, software, or combination of hardware and software used by the processing machine that allows a user to interact with the processing machine. A user interface may be in the form of a dialogue screen for example. A user interface may also include any of a mouse, touch screen, keyboard, voice reader, voice recognizer, dialogue screen, menu box, list, checkbox, toggle switch, a pushbutton or any other device that allows a user to receive information regarding the operation of the processing machine as it processes a set of instructions and/or provide the processing machine with information. Accordingly, the user interface is any device that provides communication between a user and a processing machine. The information provided by the user to the processing machine through the user interface may be in the form of a command, a selection of data, or some other input, for example.

As discussed above, a user interface is utilized by the processing machine that performs a set of instructions such that the processing machine processes data for a user. The user interface is typically used by the processing machine for interacting with a user either to convey information or receive information from the user. However, it should be appreciated that in accordance with some embodiments of the systems and methods of the invention, it is not necessary that a human user actually interact with a user interface used by the processing machine of the invention. Rather, it is also contemplated that the user interface of the invention might interact, i.e., convey and receive information, with another processing machine, rather than a human user. Accordingly, the other processing machine might be characterized as a user. Further, it is contemplated that a user interface utilized in the system and method of the invention may interact partially with another processing machine or processing machines, while also interacting partially with a human user.

The foregoing examples have been provided merely for the purpose of explanation and are in no way to be construed as limiting of the computer implemented methods and systems disclosed herein. While the computer implemented methods and systems have been described with reference to various embodiments, it is understood that the words, which have been used herein, are words of description and illustration, rather than words of limitation. Further, although the computer implemented methods and systems have been described herein with reference to particular means, materials, and embodiments, the computer implemented methods and the systems are not intended to be limited to the particulars disclosed herein—rather, the computer implemented methods and the systems extend to all functionally equivalent structures, methods and uses Those skilled in the art, having the benefit of the teachings of this specification with drawings, may affect numerous modifications thereto and changes may be made without departing from the scope and spirit of the computer implemented methods and the systems disclosed herein in their aspects.

Accordingly, it will be readily understood by those persons skilled in the art that the present invention is susceptible to broad utility and application. As noted above, many embodiments and adaptations of the present invention other than those herein described, as well as many variations, modifications and equivalent arrangements, will be apparent from or reasonably suggested by the present invention and foregoing description thereof, without departing from the substance or scope of the invention.

Accordingly, while the present invention has been described here in detail in relation to its exemplary embodiments, it is to be understood that this disclosure is only illustrative and exemplary of the present invention and is made to provide an enabling disclosure of the invention. Accordingly, the foregoing disclosure is not intended to be construed or to limit the present invention or otherwise to exclude any other such embodiments, adaptations, variations, modifications and equivalent arrangements.

1. (canceled)

2. A wearable cooling device, comprising:
a housing having a wall;
a reservoir within the housing, the reservoir being configured to hold a cooling material;
a pump within the housing; and
a conduit configured to conduct at least some of the cooling material along at least part of a flow path by which cooling is circulated by the pump from the reservoir back to the reservoir.

3. The wearable cooling device of claim 2, further comprising a controller configured to control the pump.

4. The wearable cooling device of claim 3, wherein the controller is configured to vary a rate at which heat energy is absorbed by the cooling material from a user's body.

5. The wearable cooling device of claim 4, wherein the controller is configured to vary the rate of heat energy absorption by varying a circulation rate of the cooling material.

6. The wearable cooling device of claim 2, further comprising a cooling assembly configured to control cooling of the cooling material to a temperature.

7. The wearable cooling device of claim 6, wherein the cooling assembly comprises one or more of an electrochemical link or a refrigerator.

8. The wearable cooling device of claim 7, wherein the cooling assembly is in fluid communication with the conduit between the pump and the reservoir.

9. The wearable cooling device of claim 6, wherein temperature is selected based on a freezing point of the cooling material.

10. The wearable cooling device of claim 2, wherein the wall has at least one functional layer.

11. The wearable cooling device of claim 10, wherein the layer is configured to provide at least one of comfort and safety.

12. The wearable cooling device of claim 11, wherein the safety layer is configured to prevent frostbite.

13. The wearable cooling device of claim 2, wherein the cooling material is one or more of liquid and gel.

14. The wearable cooling device of claim 3, wherein the controller is connected to a control device.

15. The wearable cooling device of claim 14, wherein the controller is connected to a control device by one or more of a wired and a wireless connection.

16. The wearable cooling device of claim 14, wherein the control device is configured to control the controller based on data input from one or more of a user device and at least one sensor component configured to be attached to a user.

17. The wearable cooling device of claim 16, wherein the sensor component is one or more of a heartrate sensor, a photocell sensor, a pressure sensor, a microphone, a thermal sensor, or an inhalation sensor.

18. The wearable cooling device of claim 16, wherein:
the sensor component comprises a heartrate sensor, a photocell sensor, a pressure sensor, a microphone, a thermal sensor, and an inhalation sensor; and
the cooling device further comprises a switch configured to control aspects of wearable power provided to the cooling device, the aspects including level of power, periodicity of power pulses, duration of power pulses, and frequency attributes of power pulses.

19. The wearable cooling device of claim 16, wherein the user device is a smartphone running a control app.

20. The wearable cooling device of claim 2, wherein the cooling device is configured to be supported by body gear including one or more of elastic band or a wearable support structure.

21. The wearable cooling device of claim 19, wherein support structure includes at least one of a vest or shirt.

22. The cooling device of claim 2, further comprising a switch configured to control aspects of wearable power provided to the cooling device, the aspects including one or more of level of power, periodicity of power pulses, duration of power pulses, or frequency attributes of power pulses.

23. The cooling device of claim 2, comprising a controller configured to perform steps for controlling the pump.

24. The cooling device of claim 2, comprising:
means for personalized circadian harmonic entrainment.
